(19) United States
(12) Patent Application Publication
(10) Pub. No.: US 2012/0095013 A1
(43) Pub. Date: Apr. 19, 2012

(54) USE OF KMUP-3 FOR MYOCARDIAL INFARCTION

(75) Inventor: Ing-Jun Chen, Kaohsiung City (TW)

(73) Assignee: KAOHSIUNG MEDICAL UNIVERSITY, Kaohsiung City (TW)

(21) Appl. No.: 13/085,017

(22) Filed: Apr. 12, 2011

(30) Foreign Application Priority Data
Oct. 18, 2010 (TW) 099135491

(51) Int. Cl. A61K 31/522; A61P 9/10; C07D 473/08

(52) U.S. Cl. 514/252.16; 544/270

(57) ABSTRACT

A pharmaceutical composition for improving a cardiac function is provided. The pharmaceutical composition comprises an effective amount of an active component being one of a KMUP-3 compound and a salt thereof.

### CROSS REFERENCE TO RELATED APPLICATIONS

This application claims the priority of TW Application No. 099135491, filed Oct. 18, 2010, which is incorporated by reference as if fully set forth herein in its entirety.

### FIELD OF THE INVENTION

The present invention relates to and used for the therapeutic function of the myocardial infarction, in particular the protection of the heart function after the myocardial infarction by KMUP-3 via the opening of the K_{ATP} channel.

### BACKGROUND OF THE INVENTION

KMUP-1 (7-[2-[4-(2-chloro benzene) piperazinyl]ethyl]-1,3-dimethylxanthine) as shown in FIG. 1 is a xanthine derivative with a theophylline backbone, which have been shown to have a therapeutic effect on the cardioprotection in hypertensive rats, the reducing of the cardiac hypertrophy and fibrosis (Wu et al., 2001; Yeh et al., 2010). The N-7 position of the backbone of the KMUP-1 is modified to have a [(2-chloro benzene) piperazinyl]ethyl group. KMUP-3 (7-[2-[4-(4-nitrobenzene)-piperazinyl]ethyl]-1,3-dimethylxanthine is a derivative of substituting the 7-2-[4-(4-nitrobenzene)-piperazinyl]ethyl group.

KMUP-3 is a phosphodiesterase inhibitor more potent than KMUP-1, and has the ability to enhance endothelial nitric oxide synthase (eNOS) in human umbilical vein endothelial cells (HUVECs) (Wu et al., 2005; Lin et al., 2006).

KMUP-3 may induce aortic smooth muscle relaxation through opening a K_{ATP} channel and increasing the eNOS. The vasorelaxant effect of KMUP-3 is attenuated by the K_{ATP} channel blocker glibenclamide to elevate the extracellular K⁺ level (80 mM) (Wu et al., 2005; Lin et al., 2006).

### SUMMARY OF THE INVENTION

Left ventricular remodelling and congestive heart failure (CHF) are still major causes of the death in survivors of acute myocardial infarction (AMI). Growing evidences indicate that the inhibition of cardiomyocyte apoptosis and the reduction of pro-inflammatory cytokines contribute to improve cardiac function after cardiac infarction (Li et al., 2009). ATP dependent potassium (K_{ATP}) channels at high levels are expressed in cardiomyocytes and exert cardioprotective effects. In acute situations, the cardioprotection resulting from the opening of K_{ATP} channels may decrease cell injury due to the ischaemia/reperfusion and reduce infarction size. Recently, long term administration of K_{ATP} channel activators has been shown to attenuate the ventricular remodelling after MI (Lee et al., 2008).

Additionally, eNOS involves in a serious of inflammatory responses by releasing the nitric oxide. Recently, transgenic mice with eNOS overexpression have been reported to have less lung injury through inhibiting the matrix metalloproteinase-9 (MMP-9) in a bleomycin-induced lung fibrosis model (Yoshimura et al., 2006). This implies that eNOS may not only involve in the acute inflammation but also have therapeutic effect on the chronic fibrosis. However, the relationship between eNOS and MMP-9 in chronic heart failure has not been established.

In view of the drawbacks of the prior arts, the inventor conceives the present invention “USE OF KMUP-3 FOR MYOCARDIAL INFARCTION” for overcoming the drawbacks of the prior arts. The summary of the present invention is described below.

The present invention provides a pharmaceutical composition for improving a cardiac function, comprising an effective amount of an active component being one of a KMUP-3 compound and a salt thereof.

According to the present invention, the pharmaceutical composition further comprises a pharmaceutically acceptable carrier.

According to the present invention, the pharmaceutical composition is used for treating a myocardial infarction, preventing a myocardial remodeling and a cardiac dysfunction after the myocardial infarction, mitigating a myocardial fibrosis and reducing a heart inflammation.

According to the present invention, the salt of the KMUP-3 compound is one selected from a group consisting of a KMUP-3-HCl salt, a KMUP-3-citric salt and a KMUP-3-nicotinic acid salt.

According to the present invention, the pharmaceutical composition reduces a protein expression of a matrix metallopeptidase-9.

According to the present invention, the pharmaceutical composition as claimed in claim 1, wherein the pharmaceutical composition increases a protein expression of an endothelial nitric-oxide synthase.

The present invention also provides a method for improving cardiac function, comprising providing an effective amount of an active component being one of a KMUP-3 compound and a salt thereof and administering the active component to a subject in need thereof.

According to the present invention, the active component is used for treating a myocardial infarction, preventing a myocardial remodeling and a cardiac dysfunction after the myocardial infarction, mitigating a myocardial fibrosis and reducing a heart inflammation.

According to the present invention, the salt of the KMUP-3 compound is one selected from a group consisting of a KMUP-3-HCl salt, a KMUP-3-citric salt and a KMUP-3-nicotinic acid salt.

According to the present invention, the subject is a patient of a myocardial infarction.

The present invention further provides a method for producing a KMUP-3 salt, comprising dissolving a KMUP compound in a solution containing a first alcohol and an acid, adding a second alcohol into the solution and isolating the KMUP-3 salt from the solution.

According to the present invention, the KMUP-3 salt is a crystal in the solution.

According to the present invention, first alcohol is an ethanol and the second alcohol is a methanol.

According to the present invention, the acid is one selected from a group consisting of an HCl, a citric acid and a nicotinic acid.

According to the present invention, the KMUP compound is dissolved at a first temperature and the second alcohol is added at a second temperature lower than the first temperature.

According to the present invention, the second temperature is a room temperature.

In the Framingham Heart Study, elevated plasma MMP-9 levels are associated with the increased left ventricular end-diastolic dimension (LVEDD) and wall thickness, indicating the potential role of MMP-9 in LV remodelling (Lin et al., 2007). Enhanced MMP-9 activity has been shown both in the acute phase of MI and the chronic heart failure. In the known studies, the targeted deletion of MMP-9 or the administration of the MMP-9 inhibitors can prevent the LV dysfunction. For these reasons, MMP-9 is considered to play a major role in the ventricular remodeling process. The MMP-9 inhibitor, tissue inhibitor of metalloproteinase-1 (TIMP-1), has been shown to be a regulator of myocardial healing in MI (Halapas et al., 2008), and a reduction of TIMP-1 exacerbates LV remodelling in MI mice (Creemers et al., 2003).

According to the experiments in the present invention, KMUP-3 compound and the salts thereof are proved to cause high level ATP dependent potassium (K_{ATP}) channels expression and exert cardioprotective effects in cardiomyocytes. In acute situations, the cardioprotection resulting from the opening of K_{ATP} channels may decrease cell injury due to the ischaemia/reperfusion and reduce infarction size. The KMUP-3 compound and the salts thereof of the present invention may be helpful to the ventricular remodelling after MI.

The above-mentioned salts include the HCl salt, the citric acid salt and the nicotinic salt formed of the KMUP-3 compound and a mineral acid or an organic acid. Specifically, KMUP-3 is dissolved in a mixture of a C₁-C₄ alcohol (i.e. the methanol and the ethanol) and a mineral acid or an organic acid to react under 40° C.-70° C. An alcohol is added into the above solution under room temperature for crystallization and a KMUP-3 salt is obtained by filtration. The above excipients or the phrases “pharmaceutically acceptable carrier or excipients” and “bio-available carriers or excipients” include any appropriate compounds known to be used for preparing the dosage form, such as the solvent, the dispersing agent, the coating, the anti-bacterial or anti-fungal agent and the preserving agent or the delayed absorbent. Usually, such kind of carrier or excipient does not have the therapeutic activity itself. Each formulation prepared by combining the derivatives disclosed in the present invention and the pharmaceutically acceptable carriers or excipients will not cause the undesired effect, allergy or other inappropriate effects while being administered to an animal or human. Accordingly, the derivatives disclosed in the present invention in combination with the pharmaceutically acceptable carrier or excipients are adaptable in the clinical usage and in the human. A therapeutic effect can be achieved by using the dosage form in the present invention by the local or sublingual administration via the venous, oral, and inhalation routes or via the nasal, rectal and vaginal routes. About 0.1 mg to 100 mg per day of the active ingredient is administered for the patients of various diseases.

The carrier is varied with each formulation, and the sterile injection composition can be dissolved or suspended in the non-toxic intravenous injection diluents or solvent such as 1,3-butanediol. Among these carriers, the acceptable carrier may be mannitol or water. Besides, the fixing oil or the synthetic glycerol ester or di-glycerol ester is the commonly used solvent. The fatty acid such as the oleic acid, the olive oil or the castor oil and the glycerol ester derivatives thereof, especially the oxy-acetylated type, may serve as the oil for preparing the injection and as the naturally pharmaceutical acceptable oil. Such oil solution or suspension may include the long chain alcohol diluents or the dispersing agent, the carboxylmethyl cellulose or the analogous dispersing agent. Other carriers are common surfactant such as Tween and Spans or other analogous emulsion, or the pharmaceutically acceptable solid, liquid or other bio-available enhancing agent used for developing the formulation that is used in the pharmaceutical industry.

The composition for oral administration adopts any oral acceptable formulation, which includes capsule, tablet, pill, emulsion, aqueous suspension, dispersing agent and solvent. The carrier generally used in the oral formulation, taking the tablet as an example, the carrier may be the lactose, the corn starch and the lubricant, and the magnesium stearate is the basic additive. The diluents used in the capsule include the lactose and the dried corn starch. For preparing the aqueous suspension or the emulsion formulation, the active ingredient is suspended or dissolved in an oil interface in combination with the emulsion or the suspending agent, and the appropriate amount of the sweetening agent, the flavors or the pigment is added as needed.

The nasal aerosol or inhalation composition may be prepared according to the well-known preparation techniques. For example, the bioavailability can be increased by dissolving the composition in the phosphate buffer saline and adding the benzyl alcohol or other appropriate preservative, or the absorption enhancing agent. The compound of the present invention may be formulated as suppositories for rectal or virginal administration.

The compound of the present invention also can be administered intravenously, as well as subcutaneously, parentally, muscular, or by the intra-articular, intracranial, intra-articular fluid and intraspinal injections, the aortic injection, the sterna injection, the intra-lesion injection or other appropriate administrations.

Other objects, advantages and efficacies of the present invention will be described in detail below taken from the preferred embodiments with reference to the accompanying drawings, in which:

### BRIEF DESCRIPTION OF THE DRAWINGS

FIG. 1 shows the structures of KMUP-1 and KMUP-3.

FIG. 2 shows the effects of KMUP-3 on the fractional shortening and the dimension of the left ventricle. FIG. 2(a) shows the fractional shortening, FIG. 2(b) shows the left ventricular dimension at the end of diastole and FIG. 2(c) shows the LV end-systolic dimension (LVESD), where symbol * denotes P<0.05 versus sham group, and symbol # denotes P<0.05 versus MI group.

FIG. 3 shows the effect of KMUP-3 on the MI size. FIG. 3(a) shows the area of risk after coronary artery ligature determined by Evans blue staining (n=5) and FIG. 3(b) shows the percentage of the infarct area (n=10), where symbol # denotes P<0.05 versus MI group.

FIG. 4 shows the percentages of the fibrosis in the myocardial infarction area. FIG. 4(a) shows the percentages of the fibrotic area in the central-infarct area (n=10), FIG. 4(b) shows the percentages of the fibrotic area in the peri-infarct area (n=10) and FIG. 4(c) shows the percentages of the fibrotic area in the non-infarct area (n=10), where symbol * denotes P<0.05 versus sham group and symbol # denotes P<0.05 versus MI group.

FIG. 5 shows the effects of KMUP-3 on levels of MMP-9 (a), tissue inhibitor of metalloproteinases-1, TIMP-1 (b) and eNOS (c) protein expression in rat hearts, determined by densitometry (n=9-10), where symbol * denotes P<0.05 versus sham group and symbol # denotes P<0.05 versus MI group.

FIG. 6 shows the levels of MMP-9 (a) and TIMP-1 (b) protein expression in human cardiac fibroblasts (HCFs), where symbol * denotes P<0.05 versus control, symbol # denotes P<0.05 versus TGF-b and symbol + denotes P<0.05 versus KMUP-3.

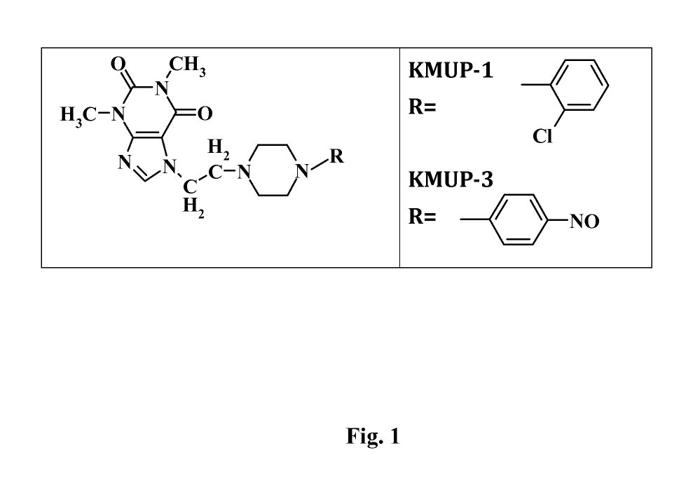

### DETAILED DESCRIPTION OF THE PREFERRED EMBODIMENT

Myocardial Infarction Experiment

Male rats (250-300 g) are provided by the National Laboratory Animal Breeding and Research Center (Taipei, Taiwan) and housed under constant temperature and controlled illumination. Food and water are available ad libitum.

MI is induced by ligation of the left anterior descending (LAD) coronary artery according to Liang et al., 2006. Briefly, under general anaesthesia with pentobarbital sodium (30 mg·kg⁻¹, i.p.), the heart is exposed via a small left thoracotomy. The LAD is ligated with 6.0 silk at 2 mm from the origin, and the wound is closed with primary suture. After the operation, the animals are observed until fully conscious. Sham animals undergo similar thoracotomy and pericardiotomy, except that the ligature around the coronary artery is not tied.

All the animals are administered with s.c. doses of analgesia (ketoprofen; 3 mg·kg⁻¹) and antibiotics (gentamicin; 0.7 mg·kg⁻¹) for 2 days. This study was approved by the Animal Care and Use Committee at Kaohsiung Medical University.

Surviving rats are fitted with ALZET osmotic minipumps (Model 2ML4, DURECT Corporation, Cupertino, Calif., USA) immediately after recovery. At this time, most of the rats are still anaesthetized from the previously administered pentobarbital sodium; a further bolus dose of pentobarbital sodium (15 mg·kg⁻¹) is given if necessary. These Alzet osmotic minipumps has a 2 mL capacity and a mean pumping rate of 2.5 mL·h⁻¹ within 4 weeks. In the treatment group, the minimumps are filled with hydrochloride of KMUP-3 (KMUP-3·HCl; 0.3 mg·kg⁻¹·day⁻¹). In the sham and MI groups, the minipumps are filled with saline.

Echocardiography is performed in all animals at the time point 4 weeks post-surgery. Each rat is anaesthetized with pentobarbital sodium (30 mg·kg⁻¹, i.p.). The anterior chest wall is shaved and acoustic coupling gel was applied. An echocardiography system (Hewlett-Packard Sonos 1500, 5-MHz probe, Andover, Mass., USA) is used through M-mode longitudinal and transverse parasternal views to measure LV end-systolic dimension (LVESD) and LVEDD. LV fractional shortening (FS) is analysed from the LV dimensions using the following formula (Louhelainen et al., 2007):

LVFS=[(LVEDD−LVESD)LVEDD]×100

After echocardiography, a PE-50 catheter is inserted through the right carotid artery to measure the LV systolic pressure (LVSP), LV end-diastolic pressure (LVEDP) and maximum rates of pressure development (LV+dP/dt) and relaxation (LV−dP/dt). At the end of the recording, the inferior vena cava and pulmonary veins are opened to avoid fluid overload. The hearts are then excised and the atria and right ventricles are dissected out. Sections of the LV are embedded in mounting medium and the remaining tissues are transferred in the liquid nitrogen for further evaluation. Throughout the whole procedure, a further bolus dose of pentobarbital sodium (15 mg·kg⁻¹) is given if required.

Histological analyses are performed to all of the heart. Rats are randomly chosen to be analyzed for area of risk after coronary artery ligature by Evan blue staining as described by Harada et al. (2005). Infarct size is determined by 2,3-,5-triphenyltetrazolium chloride (TTC) staining, and is expressed as ratio of the surface area of the infarct wall to the entire surface area of the LV (Vandegriff et al., 2008). The heart section is stained with Masson's trichrome to assess fibrosis as previously described (Lin et al., 2009; Yeh et al., 2010). The infarct area is visible as white and non-infarct as red after being stained with TTC. Peri-infarct area is defined as the region of myocardium extending 1 mm from the infarct scar. Ten sections in each heart are analyzed for the percentage of fibrosis. The mean fibrotic change after MI is compared between study groups and the Masson's trichrome staining of the sham group as control. The total LV and infarct area and fibrosis region are measured by an investigator without knowledge of the treatment group using US NIH ImageJ, 1.42q.

Human cardiac fibroblasts (HCFs; catalogue number: 306-05f) are purchased from Cell Applications Inc. (San Diego, Calif., USA). The cells are cultured as monolayer in Dulbecco's modified Eagle's medium containing 10% fetal bovine serum at 37° C. in 95% humidity and 5% CO₂. Cells are harvested at passage 3-6 for experiment. Before treatment, HCFs are washed twice with serum-free medium and switched to serum-free medium for 24 h. To stimulate MMP-9 expression, the cultures are treated with 10 ng·mL⁻¹ transforming growth factor-b (TGF-β) for 24 h. HCFs are pretreated with KMUP-3 (10 mmol·L⁻¹) or in combination with L-NAME (100 mmol·L⁻¹) for 1 h before addition of 10 ng·mL⁻¹ TGF-β for 24 h. Each experiment is repeated three times.

Western Blot Analysis

The hearts are sonicated in 50 mmol·L⁻¹ Tris for 10 s two times and centrifuged at 13,000 rpm at 4° C. for 30 min. The protein concentration of the supernatants is determined by using bovine serum albumin as the standard. The cell extracts are boiled in ratio of 4:1 with sample buffer (100 mmol·L⁻¹ Tris, pH 6.8, 20% glycerol, 4% SDS, and 0.2% bromophenol blue). Electrophoresis is performed using 10% SDS-polyacrylamide gel electrophoresis and transferred to nitrocellulose membranes (Millipore Corporation, Billerica, Mass., USA). The membranes are blocked with Tris-buffered saline (20 mmol·L⁻¹ Tris and 137 mmol·L⁻¹ NaCl, pH 7.6) containing 0.1% Tween-20 (TTBS) and 5% nonfat milk at room temperature for 1 h, washed with TTBS, and then incubated overnight at 4° C. with MMP-9, TIMP-1 (Millipore, Temecula, Calif., USA), or eNOS (BD Transduction Laboratories, Franklin Lakes, N.J., USA) primary antibody. The membranes are washed in TTBS before being incubated with horseradish peroxidase-conjugated antibody against mouse or rabbit IgG (Santa Cruz Biotechnology, Santa Cruz, Calif., USA) for 1 h. The membranes are then washed in TTBS again and developed with the enhanced chemiluminescence for the detection of the specific antigen. The intensity of the bands is quantified by densitometry.

Early surgery-related death assigned to severe thoracic bleeding within 6 h after surgery was similar in all groups (9%) and these rats were therefore excluded from final analysis. None of the animals died during the post-MI treatment period. As indicated in Table 1, MI induces significant cardiac hypertrophy with an increased heart weight to body weight ratio. KMUP-3 Treatment prevents the cardiac remodelling process. The LV+dP/dt decreased in the MI group, while administration of KMUP-3 (0.3 mg·kg−1·day−1) for 4 weeks improved cardiac systolic function. The LVSP and LV−dP/dt also tends to be decreased in the MI group but none of these differences reached statistical significance. A small increase in heart rate occurrs after KMUP-3 treatment, but this is not statistically significant.

TABLE 1
Effects of KMUP-3 on cardiac structure and haemodynamics

| | Sham | MI | MI + KMUP-3 |
|---|---|---|---|
| n | 10 | 10 | 10 |
| HW(mg) | 1228 ± 72 | 1300 ± 32 | 1219 ± 39 |
| BW (g) | 446 ± 30 | 441 ± 10 | 448 ± 10 |
| HW/BW (mg/g) | 2.76 ± 0.05 | 2.96 ± 0.06 | 2.72 ± 0.04* |
| LVSP(mmHg) | 132.1 ± 11.6 | 117.6 ± 7.4 | 140.6 ± 4.2 |
| LVEDP(mmHg) | 9.3 ± 0.7 | 12.1 ± 1.7 | 12.6 ± 2.3 |
| LV +dP/dt | 7348 ± 986 | 4565 ± 394 | 7637 ± 333** |
| (mmHg/s) | | | |
| LV −dP/dt | −5633 ± 911 | −3603 ± 353 | −5379 ± 441 |
| (mmHg/s) | | | |
| HR (bpm) | 370 ± 21 | 380 ± 22 | 421 ± 5 |

+dP/dt and −dP/dt, maximum rate of rise and full of pressure; HR, heart rate; HW, heart weight.
*denotes P < 0.05 versus sham.
#denotes P < 0.05.
##denotes P < 0.01 versus MI rats.

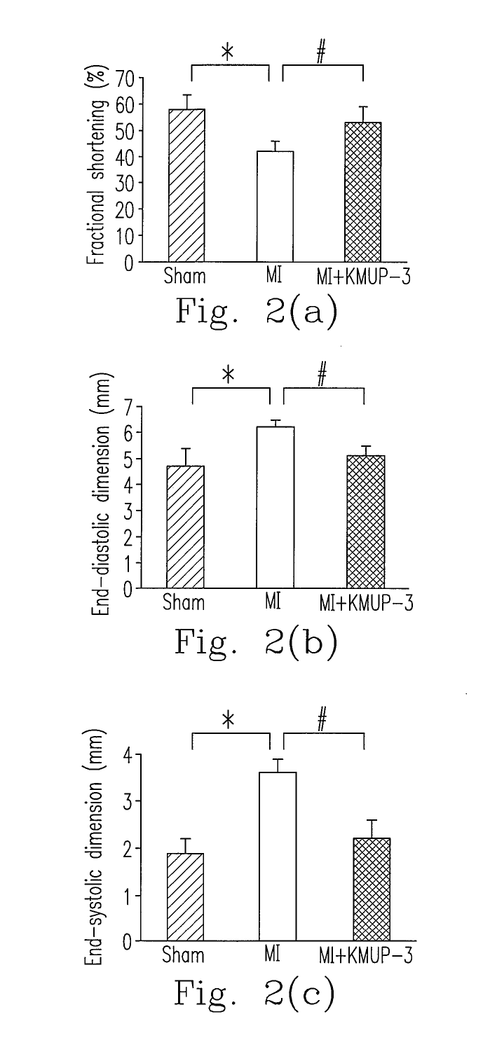

As FIGS. 2(b)-(c) show, the LVEDD and LVESD of MI rats are increased while being compared with the sham-operated group. The decreased systolic function is also demonstrated by the decrease in FS in the MI group (FIG. 2(a)). The cardiac remodeling and dysfunction may be prevented by the administration of KMUP-3 for 4 weeks.

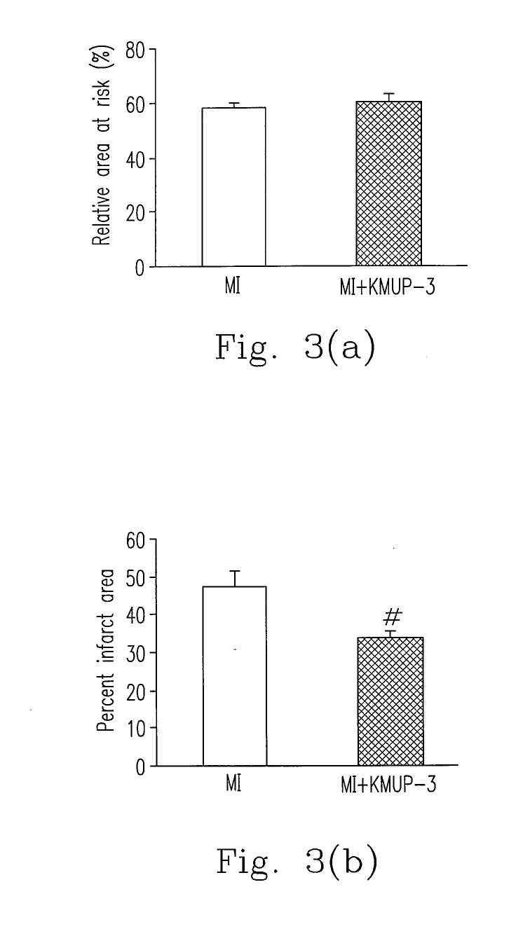

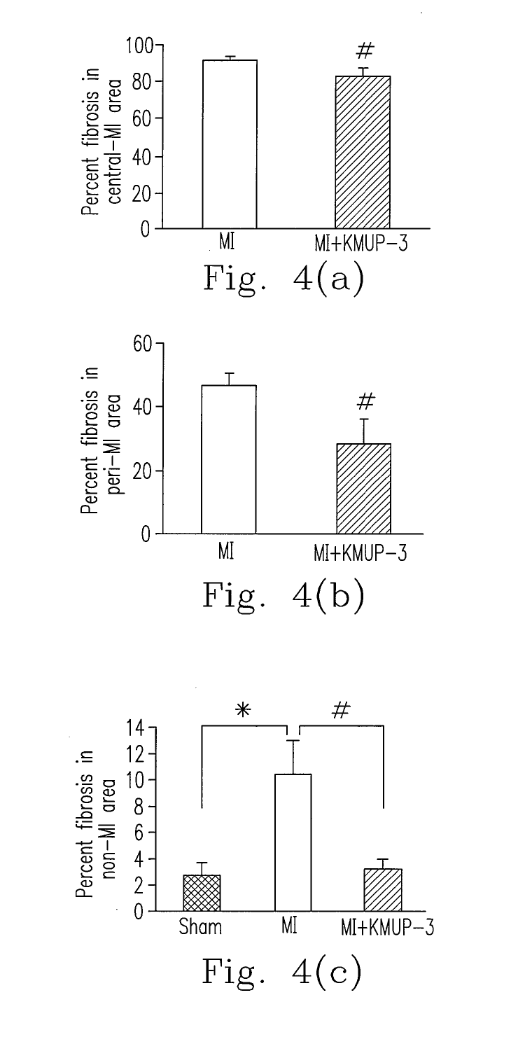

The area of risk after coronary artery ligature is determined by Evans blue staining, and there is no difference between treatment groups (FIG. 3a). Infarction size, determined by TTC staining, decreases significantly after KMUP-3 treatment (47.4±3.7% vs. 33.6±1.7%, respectively, P<0.05) (FIG. 3b). KMUP-3 attenuates cardiac fibrosis in both the central-infarct and peri-infarct areas as determined by Masson's trichrome staining. The anti-fibrotic effect of KMUP-3 is also found in the non-infarcted area, as the percentage of fibrosis is comparable with that of the sham-operated hearts.

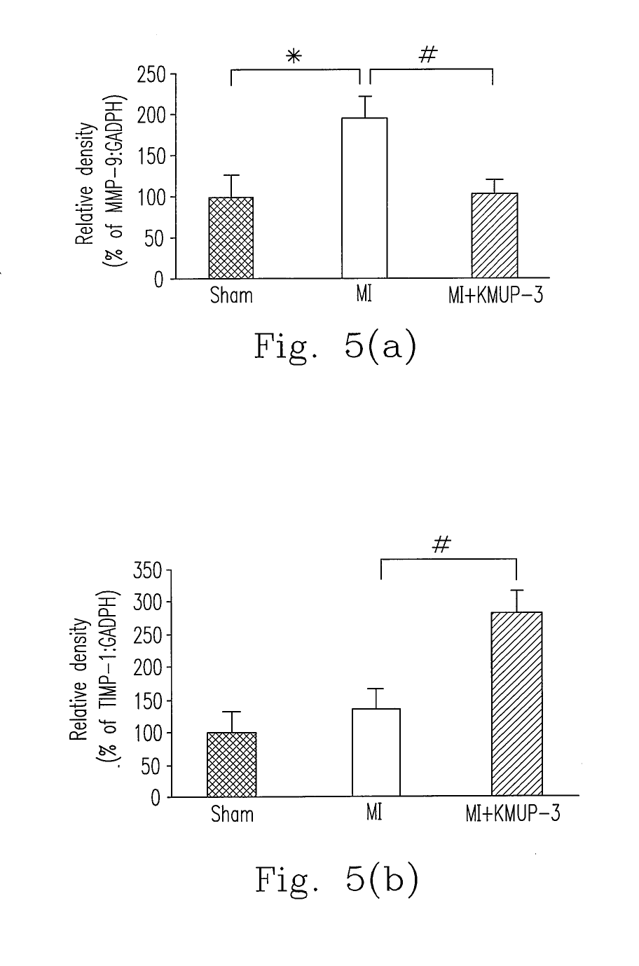

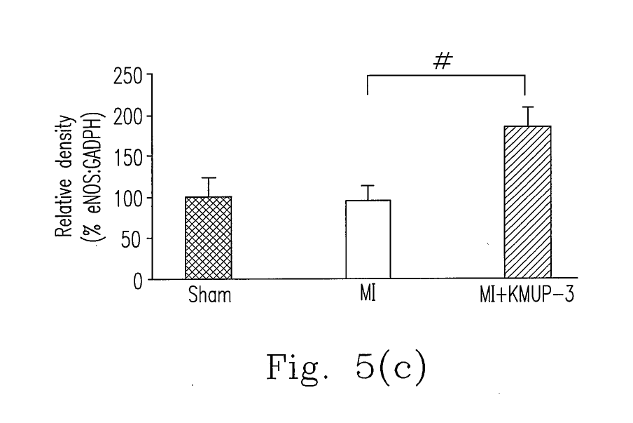

MMP-9 expression increases significantly in MI rats, compared with sham-operated rats. The administration of KMUP-3 decreases MMP-9 expression in MI rats (FIG. 5a). It is also noted that the expression of the MMP-9 inhibitor TIMP-1 increases significantly after treatment with KMUP-3 (FIG. 5b). A small decrease in eNOS expression is noted in MI rats. After treatment with KMUP-3, the eNOS expression increases significantly than rats in the MI group (FIG. 5c).

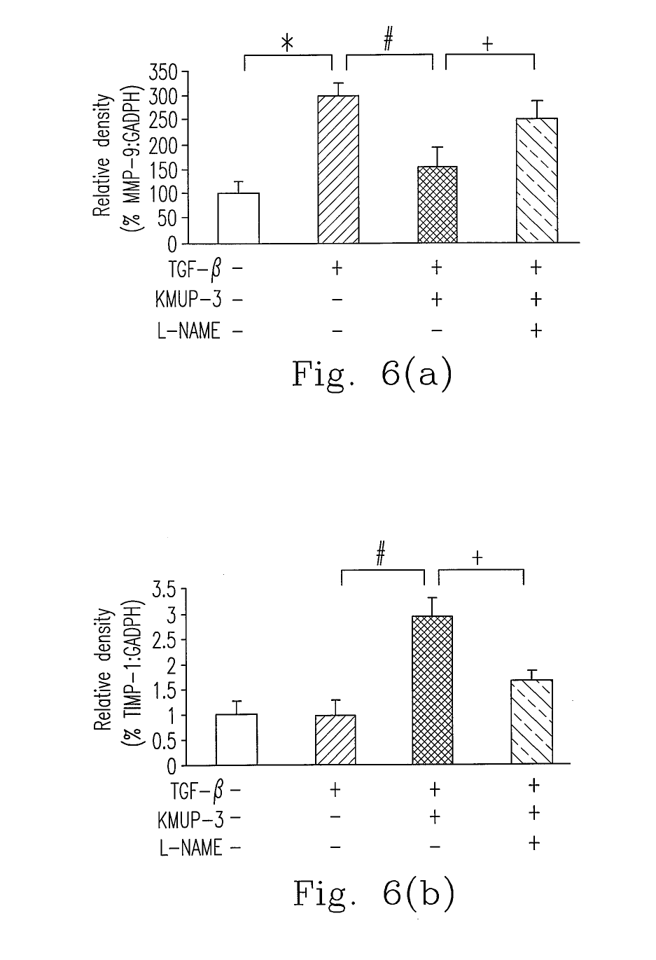

To investigate the mechanism of eNOS dependent cardiac protection in vitro, HCFs are stimulated with TGF-β (10 ng·mL⁻¹) and the expression of MMP-9 and TIMP-1 are measured. MMP-9 expression increases significantly following TGF-b stimulation (FIG. 6a) and the administration of KMUP-3 (10 mmol·L⁻¹) attenuates the expression of MMP-9. Pretreatment with the eNOS inhibitor N^{ω}-nitro-L-arginine methyl ester (L-NAME; 100 mmol·L⁻¹) reverses the inhibition of MMP-9 expression (FIG. 6a). At the same time, TIMP-1 expression is significantly enhanced by the treatment with KMUP-3 (FIG. 6b).

### Example 1

### Preparation of KMUP-3HCl salt (7-[2-[4-(4-nitrobenzene)piperazinyl]ethyl]-1,3-dimethyl xanthine HCl)

KMUP-3 (8.3 g) is dissolved in a mixture of ethanol (10 mL) and 1N HCl (60 mL) The solution is reacted at 50° C. for 20 mins, the ethanol (or methanol) is added thereinto under room temperature and the solution is incubated over night for crystallization and filtrated to obtain KMUP-3HCl salt (6.4 g).

### Example 2

### Preparation of KMUP-3-Citric Acid Salt

KMUP-3 (8.3 g) is dissolved in a mixture of ethanol (10 mL) and citric acid (4 g) and reacted at 50° C. for 20 min, the ethanol (or methanol) is added thereinto under room temperature and the solution is incubated over night for crystallization and filtrated to obtain KMUP-3 citric acid salt (10.5 g).

### Example 3

### Preparation of KMUP-3-Nicotinic Acid Salt

KMUP-3 (8.3 g) is dissolved in a mixture of ethanol (10 mL) and nicotinic acid (2.4 g) and reacted at 50° C. for 20 min, the ethanol (or methanol) is added thereinto under room temperature and the solution is incubated over night for crystallization and filtrated to obtain KMUP-3 nicotinic acid salt (8.3 g).

### Example 4

### Preparation of the Composition in Tablet

The following components are weighted respectively and filled into a tabletting machine after mixing for preparing tablets.

| | |
|---|---|
| KMUP-3 HCl | 0.12 g |
| Lactose | qs |
| Corn starch | qs |

### REFERENCE

1. Creemers E E, Davis J N, Parkhurst A M, Leenders P, Dowdy K B, Hapke E, et al. (2003). Deficiency of TIMP-1 exacerbates LV remodeling after MI in mice. Am J Physiol Heart Circ Physiol 284: H364-371.
2. Halapas A, Zacharoulis A, Theocharis S, Karavidas A, Korres D, Papadopoulos K, et al. (2008). Serum levels of the osteoprotegerin, receptor activator of nuclear factor kappa-B ligand, metalloproteinase-1 (MMP-1) and tissue inhibitors of MMP-1 levels are increased in men 6 months after acute MI. Clin Chem Lab Med 46: 510-516.
3. Harada M, Qin Y, Takano H, Minamino T, Zou Y, Toko H et al. (2005). G-CSF prevents cardiac remodeling after myocardial infarction by activating the Jak-Stat pathway in cardiomyocytes. Nat Med 11: 305-311.
4. Lee T M, Lin M S, Chang N C (2008). Effect of ATP-sensitive potassium channel agonists on ventricular remodeling in healed rat infarcts. J Am Coll Cardiol 51: 1309-1318.
5. Li G H, Shi Y, Chen Y, Sun M, Sader S, Maekawa Y, et al. (2009). Gelsolin regulates cardiac remodeling after myocardial infarction through DNase I-mediated apoptosis. Circ Res 104: 896-904.
6. Liang J C, Chen H R, Chiu C C, Liou S F, Chen I J, Yeh J L (2006). Protective effect of labedipinedilol-A, a novel dihydropyridine-type calcium channel blocker, on myocardial apoptosis in ischemiareperfusion injury. Life Sci 79: 1248-1256.
7. Lin R J, Wu B N, Lo Y C, An L M, Dai Z K, Lin Y T, et al. (2006). A xanthine-based epitheliumdependent airway relaxant KMUP-3 (7-[2-[4-(4-nitrobenzene)piperazinyl]ethyl]-1,3-dimethylxanthine) increases respiratory performance and protects against tumor necrosis factoralpha-induced tracheal contraction, involving nitric oxide release and expression of cGMP and protein kinase G. J. Pharmacol Exp Ther 316: 709-717.
8. Lin T H, Chiu H C, Lee Y T, Su H M, Juo S H, Voon W C, et al. (2007). The C-allele of tissue inhibitor of metalloproteinases 2 is associated with increased magnitude of QT dispersion prolongation in elderly Chinese—4-year follow-up study. Clin Chim Acta 386: 87-93.
9. Lin X, Jo H, Ishii T M, Fujita M, Fu M, Tambara K, et al. (2009). Controlled release of matrix metalloproteinase-1 plasmid DNA prevents left ventricular remodeling in chronic myocardial infarction of rats. Circ J 73: 2315-2321.
10. Louhelainen M, Vahtola E, Kaheinen P, Leskinen H, Merasto S, Kytö V, et al. (2007). Effects of levosimendan on cardiac remodeling and cardiomyocyte apoptosis in hypertensive Dahl/Rapp rats. Br J Pharmacol 150: 851-861.
11. Vandegriff K D, Young M A, Lohman J, Bellelli A, Samaja M, Malavalli A, et al. (2008). CO-MP4, a polyethylene glycol-conjugated haemoglobin derivative and carbon monoxide carrier that reduces myocardial infarct size in rats. Br J Pharmacol 154: 1649-1661.
12. Wu B N, Lin R J, Lin C Y, Shen K P, Chiang L C, Chen I J (2001). A xanthine-based KMUP-1 with cyclic GMP enhancing and K (+) channels opening activities in rat aortic smooth muscle. Br J Pharmacol 134: 265-274.
13. Wu B N, Chen I C, Lin R J, Chiu C C, An L M, Chen I J (2005). Aortic smooth muscle relaxants KMUP-3 and KMUP-4, two nitrophenylpiperazine derivatives of xanthine, display cGMP-enhancing activity: roles of endothelium, phosphodiesterase, and K+ channel. J Cardiovasc Pharmacol 46(5): 600-608.
14. Yeh J L, Hsu J H, Wu P J, Liou S F, Liu C P, Chen I J, et al. (2010). KMUP-1 attenuates isoprenalineinduced cardiac hypertrophy in rats through NO/cGMP/PKG and ERK1/2/calcineurin A pathways. Br J Pharmacol 159: 1151-1160.
15. Yoshimura S, Nishimura Y, Nishiuma T, Yamashita T, Kobayashi K, Yokoyama M (2006). Overexpression of nitric oxide synthase by the endothelium attenuates bleomycin-induced lung fibrosis and impairs MMP-9/TIMP-1 balance. Respirology 5: 546-556.

What is claimed is:

1. A pharmaceutical composition for improving a cardiac function, comprising:
an effective amount of an active component being one of a KMUP-3 compound and a salt thereof.

2. A pharmaceutical composition as claimed in claim 1, further comprising a pharmaceutically acceptable carrier.

3. A pharmaceutical composition as claimed in claim 1, wherein the pharmaceutical composition is used for treating a myocardial infarction, preventing a myocardial remodeling and a cardiac dysfunction after the myocardial infarction, mitigating a myocardial fibrosis and reducing a heart inflammation.

4. A pharmaceutical composition as claimed in claim 1, wherein the salt of the KMUP-3 compound is one selected from a group consisting of a KMUP-3-HCl salt, a KMUP-3-citric salt and a KMUP-3-nicotinic acid salt.

5. A pharmaceutical composition as claimed in claim 1, wherein the pharmaceutical composition reduces a protein expression of a matrix metallopeptidase-9.

6. A pharmaceutical composition as claimed in claim 1, wherein the pharmaceutical composition increases a protein expression of an endothelial nitric-oxide synthase.

7. A method for improving cardiac function, comprising the following steps:
providing an effective amount of an active component being one of a KMUP-3 compound and a salt thereof; and
administering the active component to a subject in need thereof.

8. A method as claimed in claim 7, wherein the active component is used for treating a myocardial infarction, preventing a myocardial remodeling and a cardiac dysfunction after the myocardial infarction, mitigating a myocardial fibrosis and reducing a heart inflammation.

9. A method as claimed in claim 7, wherein the salt of the KMUP-3 compound is one selected from a group consisting of a KMUP-3-HCl salt, a KMUP-3-citric salt and a KMUP-3-nicotinic acid salt.

10. A method as claimed in claim 7, wherein the subject is a patient of a myocardial infarction.

11. A method for producing a KMUP-3 salt, comprising the following steps:
dissolving a KMUP compound in a solution containing a first alcohol and an acid;
adding a second alcohol into the solution; and
isolating the KMUP-3 salt from the solution.

12. A method as claimed in claim 11, wherein the KMUP-3 salt is a crystal in the solution.

13. A method as claimed in claim 11, wherein the first alcohol is an ethanol and the second alcohol is one of an ethanol and a methanol.

14. A method as claimed in claim 11, wherein the acid is one selected from a group consisting of an HCl, a citric acid and a nicotinic acid.

15. A method as claimed in claim 11, wherein the KMUP compound is dissolved at a first temperature and the second alcohol is added at a second temperature lower than the first temperature.

16. A method as claimed in claim 15, wherein the second temperature is a room temperature.
